(19) United States
(12) Patent Application Publication
(10) Pub. No.: US 2003/0068324 A1
(43) Pub. Date: Apr. 10, 2003

(54) Conjugate vaccine composed of the polysaccharide moiety of the lipopolysaccharide of Vibrio cholerae O139 bound to tetanus toxoid

(76) Inventors: Jean-Michel Fournier, Paris (FR); Alain Boutonnier, Paris (FR)

Correspondence Address: Finnegan, Henderson, Farabow,, Garrett & Dunner, L.L.P., 1300 I Street, N.W., Washington, DC, 20005-3315, US

(21) Appl. No.: 10/116,109

(22) Filed: Apr. 5, 2002

Related U.S. Application Data
(60) Provisional application No. 60/281,783, filed on Apr. 6, 2001.

(51) Int. Cl. A61K 39/00; A61K 39/38

(52) U.S. Cl. 424/184.1

(57) ABSTRACT

The epidemic and pandemic potential of V. cholerae O139 is such that a vaccine against this newly emerged serogroup of V. cholerae is required. A conjugate made of the polysaccharide moiety (O-specific polysaccharide+core) of the lipopolysaccharide (LPS) of V. cholerae O139 (pmLPS) was prepared by derivatization of the pmLPS with adipic acid dihydrazide and coupling to tetanus toxoid (TT) by carbodiimide-mediated condensation. The immunologic properties of the conjugate were tested using BALB/c mice injected subcutaneously three times at 2 weeks interval and then a fourth time 4 weeks later. Mice were bled 7 days after each injection and then once each month for the following six months. LPS and TT antibody levels were determined by ELISA using immunoplates coated with either O139 LPS or TT. Both pmLPS and pmLPS-TT conjugate elicited low levels of IgM, peaking 5 weeks after the first immunization. The conjugate elicited high levels of IgG antibodies, peaking 3 months after the first immunization and declining slowly during the following 5 months. TT alone, or as a component of conjugate, induced mostly IgG antibodies. Antibodies elicited by the conjugate recognized both capsular polysaccharide (CP) and LPS from V. cholerae O139, and were vibriocidal. They were also protective in the neonatal mouse model of cholera infection. The conjugation of the O139 pmLPS, therefore, enhanced its immunogenicity and conferred T-dependent properties to this polysaccharide.

This application is based on and claims the benefit of priority of U.S. Provisional Application Serial No. 60/281,783, filed Apr. 6, 2001 (Attorney Docket No. 03495-6067). The entire disclosure of this application is relied upon and incorporated by reference.

Since the appearance of Vibrio cholerae O139 in the suburb of Madras in October 1992, epidemic cholera caused by this strain has spread rapidly throughout the Indian subcontinent (1). Clinical illness associated with V. cholerae O139 infection appears to be virtually identical to that due to V. cholerae O1 El Tor infections. However, in contrast to infection with V. cholerae O1, V. cholerae O139 infection has largely affected the adult population in V. cholerae O1 endemic areas, indicating a lack of protective immunity against this newly evolved strain (1). Presumably, there are differences between the immune responses against O1 and O139 strains, which may be of considerable importance in terms of protection (33). A quiescent period followed the appearance of V. cholerae O139 and it was thought that it was a one-time event. However, there was an upsurge of cases in Calcutta in 1996 and the O139 serogroup again became the dominant serogroup causing cholera in India by September 1996 (32). The O139 serogroup has remained present in India and Bangladesh since this last outbreak (15) and requires careful monitoring.

The epidemic and pandemic potential of V. cholerae O139 poses a serious threat to developing countries, and a vaccine against this novel strain is therefore required. The absence of cross-protection between V. cholerae O1 and V. cholerae O 139 serogroups, documented in rabbits either immunized with live bacteria (2) or passively protected with sera of convalescent cholera patients (33), suggested that protection against cholera is LPS-specific. This is supported by the correlation observed between the protective effect of rabbit O139 antisera and anti-LPS Abs titers (25).

It has been suggested that the emergence of V. cholerae O139 is the result of a complex chromosomal rearrangement involving the horizontal transfer of genes encoding enzymes involved in O-specific polysaccharide (O-SP) biosynthesis (3, 8, 14, 43). Indeed, the major differences betwen V. cholerae O1 and V. cholerae O139 reside in their cell surface components. V. cholerae O139, unlike V. cholerae O1, expresses capsular polysaccharide (CP) (43, 46). Both the structure of the CP and that of the lipopolysaccharide (LPS) from V. cholerae O139 have been characterized (FIG. 1) (11, 12, 28, 36). Although, O139 LPS and CP share the same repeat unit, only the CP is polymerized (12). Nevertheless, CP and LPS share common epitopes (43).

Several oral cholera vaccines, either inactivated or live attenuated, have been developed to elicit protection against this new serogroup of V. cholerae (10, 23, 40, 44). Various subcellular fractions of V. cholerae O139 administered subcutaneously have been evaluated in the rabbit ileal loop model of experimental cholera, and the immune response directed against the O139 serogroup antigen appeared to be determinant for protective immunity (4). It has been proposed that serum IgG antibodies (Abs) confer protection against enteric diseases by inactivating the inoculum on the mucosal surfaces (38). Systemic administration of IgG Abs specific for the O-SP of V. cholerae O1 was found to protect neonatal mice against loss of weight and death following intragastral challenge with V. cholerae O1 (5). A V. cholerae O139 CP-tetanus toxoid conjugate vaccine induced protection in the rabbit ileal loop model of experimental cholera (24). More recently, V. cholerae O139 CP conjugated with a recombinant mutant diphtheria toxin was shown to elicit high levels of serum anti-CP IgG in mice with vibriocidal activity (30). Other vaccines based on polysaccharide-protein conjugate to prevent cholera were developed (16, 17).

In this study, a conjugate prepared with the polysaccharide moiety (O-SP+core) of the LPS (pmLPS) from V. cholerae O139 bound to tetanus toxoid (TT) has been synthesized. The synthesis, characterization and immunologic properties in mice of this conjugate were assessed.

Said conjugate vaccine elicits anti-O139 Abs in mice; the immunologic properties of these Abs were also studied. As observed in many LPS from various Gram-negative bacteria, the V. cholerae pmLPS is attached to the lipid A portion of the molecule through Kdo (12, 48). This bond is cleaved by mild acid hydrolysis (FIG. 1) to release a polysaccharide bearing a Kdo residue at its reducing end (22). The use of the carboxylic group of the Kdo moiety for polysaccharide-protein coupling results in a saccharide with a single terminal active site for conjugation. This single-end activated pmLPS showed a high potential for use as a vaccine: (i) the O139 specific antigenic determinant(s) are conserved; (ii) it is the simplest conjugate configuration in which polysaccharide chains radiate from the protein carriers; (iii) the coupling procedure is the easiest to control, producing well-defined non-crosslinked, water soluble conjugate molecules of known configuration (22).

It has been shown that phenol-water extraction of capsulated bacteria yields a mixture of LPS and CP in the aqueous phase (11, 28, 37, 46). To confirm the effective separation of LPS from CP after further purification steps, these two types of cell surface polysaccharide were identified by tricine SDS-PAGE using differential staining. Only the rapidly migrating material, corresponding to LPS, was silver stained, but the slowly migrating forms of the O139 antigen were not. This result is consistent with the previous observation that O139 CP is not stained with silver (34). It is thought that the silver staining of polysaccharides depends on the presence of periodate-sensitive cis-hydroxyl groups in the monosaccharide residues (9). Thus, as the O139 CP repeating unit, unlike the LPS core, lacks cis-hydroxyls (11, 12, 28, 36) it is not silver stained. However, this CP, which is acidic, is stained by the cationic dye, Alcian Blue.

The various aspects of the present invention are based upon the discovery of the properties of the polysaccharide moiety (O-SP+core), also named pmLPS, of the LPS from V. cholerae O139 and more specially of a conjugate prepared with this polysaccharide moiety bound to tetanus toxoid (TT).

Therefore, the invention provides an immunogenic composition against Vibrio infection comprising an O-SP unit of LPS of Vibrio associated to a core molecule of LPS of Vibrio or a polymer of said composition.

As used herein, a “polymer” of the composition of the invention means a composition comprising several, at least two, O-SP+core (pmLPS) linked by any means one to another or together.

According to a preferred embodiment of said immunogenic composition, the O-SP unit associated to the core molecule of LPS of Vibrio is part of a conjugate further comprising a carrier protein.

Carrier proteins are known form the one skilled in the art . Examples of bacterial carrier proteins are diphteria toxins, tetanus toxoïd . . .

Preferably:

the Vibrio O-SP unit and core molecule are bound to the carrier protein of the conjugate by a covalent link.

the carrier protein is a bacterial protein, for instance tetanus toxoid.

According to another preferred embodiment of said immunogenic composition, it further comprises an adjuvant and/or a pharmaceutically acceptable carrier.

Adjuvants and pharmaceutically acceptable carriers are known from the one skilled in the art.

Examples of species from Vibrionacae family are: V. alginolyticus, V. cholerae , V. cincinnatiensis, V. diabolicus, V. diazotrophicus, V. harveyi, V. logei, V. natriegens, V. nereis, V. splendidus, V. tubiashii, V. halioticoli, V. ichthyoenteri, V. pectenicida and V. wodanis.

According to yet another preferred embodiment of said immunogenic composition, the LPS is from Vibrio cholera, more preferably from Vibrio cholera serogroup O139.

According to yet another preferred embodiment of said immunogenic composition, the O-SP unit and the core are from two different Vibrio.

The present invention also includes a vaccine composition comprising an O-SP unit of LPS of Vibrio associated to a core molecule of LPS of Vibrio or a polymer of said composition, said vaccine composition being protective against infection from Vibrio.

According to a preferred embodiment of said vaccine composition, it is protective against infection from Vibrio cholerae , preferably against infection from Vibrio cholera serogroup O139.

The invention further includes a method for preparing a conjugate as defined above, i.e. comprising an O-SP unit associated to the core molecule of LPS of Vibrio and a carrier protein, comprising:

a) providing LPS from Vibrio;

b) hydrolyzing the lipid A-core linkage for obtaining an O-SP unit associated to a core molecule;

c) derivatizing the O-SP unit associated to the core molecule of step b);

d) bounding the derivatized the O-SP unit associated to the core molecule of step c) to a carrier protein;

e) collecting the O-SP unit associated to the core molecule bound to the carrier protein in step d).

According to said method, the O-SP unit associated to the core molecule is bound to the carrier protein by a covalent link.

More preferably, in said method:

the carrier protein is a bacterial protein.

the bacterial toxin is tetanus toxoid.

LPS of step a) is from Vibrio cholerae , more preferably from Vibrio cholerae serogroup O139.

The derivatization ratio of pmLPS, an essential step in the coupling procedure of the invention, was lower than usual with other polysaccharides (13, 16). Nevertheless, the polysaccharide/protein ratio (0.99 mol/mol) obtained herein was sufficient for a strong IgG response in immunized mice. The unconjugated pm-LPS elicited mostly IgM Abs, whereas only low levels of IgG anti-LPS Abs were detected. This response was similar to those previously reported for polysaccharides tested in mice (45). In contrast, the pmLPS-TT conjugate elicited mostly IgG anti-LPS Abs, which were boosted following reimmunization. Moreover, after the fourth immunization, a high level of these IgG Abs was maintained for 5 months. It was found that pm-LPS-TT had typical T-dependent properties. Similar results have been obtained with O-SP from several other enteric bacterial pathogens (7, 29).

Interestingly, Abs obtained in mice immunized with pmLPS conjugated to TT recognized both O-SP and CP purified from V. cholerae O139. This result is entirely consistent with the observation that CP and LPS share common epitope(s) expressed by a common hexasaccharide unit (12, 43). This cross-reactivity between O139 pmLPS and CP accounts for the finding of the invention that pmLPS-TT Abs reacted with both encapsulated and non-encapsulated V. cholerae O139 strains, and is consistent with observations that protection against V. cholerae O139 can be mediated by Abs directed against either the LPS or CP of this novel cholera vibrio (24, 25, 33, 34, 39). The invention demonstrates the efficiency of a conjugated pmLPS in eliciting an IgG response in mice and justifies clinical evaluation of this V. cholerae O139 conjugate.

The invention also includes a method for immunizing human or animal against Vibrio infection, wherein said method comprises administration to said human or animal of a composition as defined hereabove, wherein Vibrio infection is preferably an infection from Vibrio cholerae and more preferably from Vibrio cholerae serogroup O139.

Therefore, the invention also includes the use of a composition comprising a conjugate compound comprising an O-SP unit of LPS of Vibrio associated to a core molecule of LPS of Vibrio bound to a protein carrier for the preparation of a medicament for preventing a Vibrio infection, more preferably a Vibrio infection from Vibrio cholerae and more preferably from Vibrio cholerae serogroup O139.

The instant invention further includes a conjugate compound comprising an O-SP unit of LPS of Vibrio associated to a core molecule of LPS of Vibrio bound to a protein carrier.

According to a preferred embodiment of said conjugate, the Vibrio O-SP unit associated to the Vibrio core molecule is bound to the protein carrier by a covalent link.

According to another preferred embodiment of said conjugate, the protein carrier is a bacterial protein, more preferably tetanus toxoid.

According to yet another embodiment of said conjugate, the Vibrio LPS is from Vibrio cholerae , more preferably from Vibrio cholerae serogroup O 139.

According to yet another embodiment of said conjugate, the O-SP unit and the core are from two different Vibrio.

The present invention will be further illustrated by the additional description which follows, which refers to examples of preparation and use of the conjugate according to the invention.

It should be clearly understood however that these examples are given solely as illustration of the subject of the invention and do not constitute in any manner a limitation thereof.

### DESCRIPTION OF THE DRAWINGS

FIG. 1. Overall structure of the LPS of V. cholerae O139. The O-specific polysaccharide (O-SP) and the core structure are taken from Cox et al. (11, 12) and the lipid A structure is arranged according to Kabir (26) and Wilkinson (48). The arrow indicates the lipid A-core bond hydrolyzed by acetic acid treatment: this treatment releases the polysaccharide moiety (O-SP+core) of the LPS (pmLPS).

FIG. 2. Analysis of polysaccharide preparations of V. cholerae O139. (A) Tricine SDS-PAGE (16.5%). The gel was stained with silver. (B) SDS-PAGE (10%). The gel was pretreated with Alcian Blue, a cationic dye that binds acidic polysaccharides, prior to silver staining. (C) Immunoblot analysis with hyperimmune O139 mice antiserum as the probe. Mᵣ values are shown on the left. MF: migration front.

FIG. 3. Double immunodiffusions. A, mAb anti-LPS O139; 1, pmLPS O139; 2, LPS O139; 3, CP O139; 4, LPS O1; 5, derivatized pmLPS O139; 6, pmLPS-TT.

FIG. 4. Time course of amounts of IgM (▪) and IgG (●) anti-O139 Abs, and O139 vibriocidal Abs titer (▴) in serum of a single mouse immunized four times (arrows) with pmLPS-TT.

FIG. 5. Protective activity of anti-pmLPS-TT Abs against challenge with 10×LD₅₀ of V. cholerae O139 in the suckling-mouse model: NI, pooled non immune sera; IS, pooled immune sera obtained on days 152 and 231 from mice immunized with pmLPS-TT. Health status was scored 48 h after challenge.

### EXAMPLE 1

### Preparation and Characterization of LPS, pmLPS, and CP.

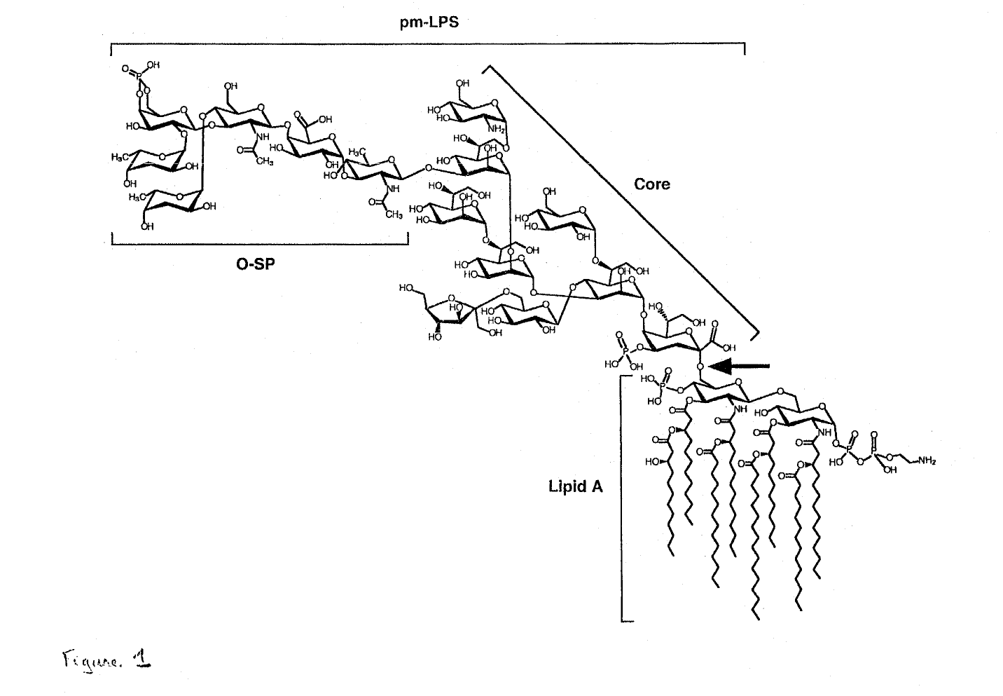

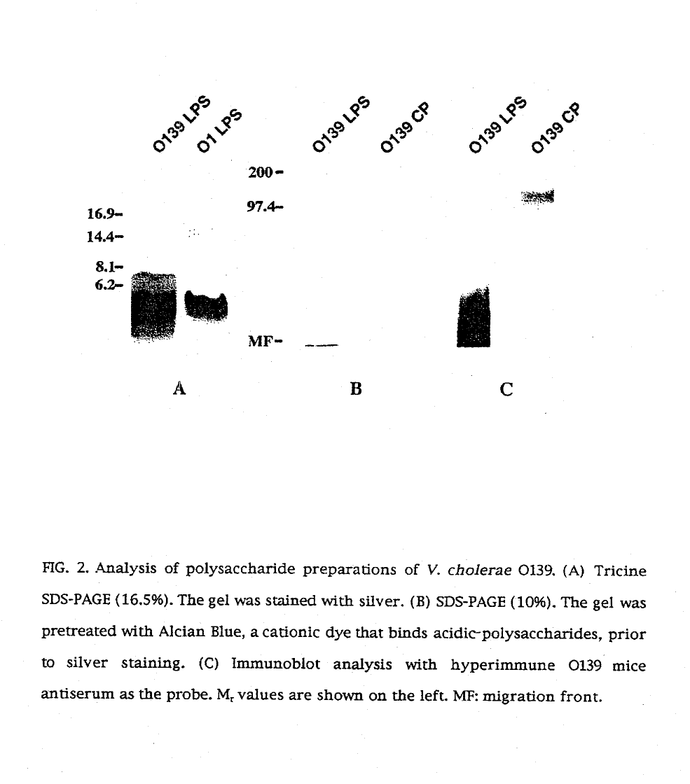

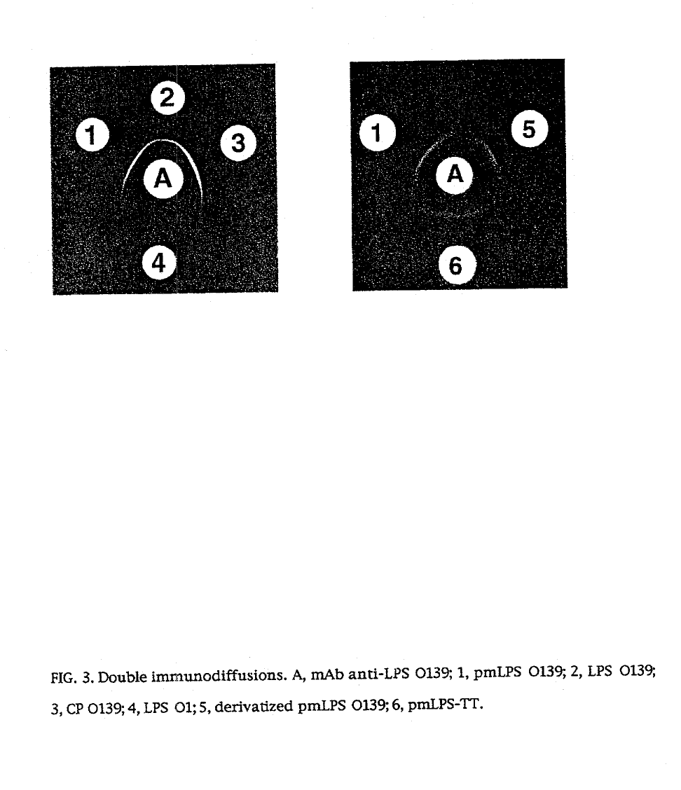

V. cholerae O139 (strain MO45, kindly provided by Y. Takeda, Kyoto University, Japan) was grown in Tryptic Soy agar (Difco) at 37° C. for 18 h. LPS was obtained by hot phenol water extraction (47), followed by enzymatic treatment (DNase, RNase and protease) and ultracentrifugation. The pellet, containing the LPS had 0.5% (w/v) protein and less than 0.2% (w/v) nucleic acid. LPS was treated with acetic acid to hydrolyze the lipid A-core linkage (FIG. 1) (19). The resulting product is referred to as pmLPS. For the preparation of CP, LPS was removed from the ultracentrifugation supernatant by passage through a Sephacryl S-200 column in a buffer containing deoxycholic acid (37). Void volume fractions containing CP, detected by refractive index and 10% SDS-PAGE in gels treated with Alcian Blue (a cationic dye that binds acidic polysaccharides) prior to silver staining (9), were dialyzed extensively against 10% (v/v) ethanol to remove deoxycholic acid (37). The LPS had 2×10⁴ endotoxin units/μg and the pmLPS had 10 endotoxin units/μg as assessed by the Limulus amebocyte lysate assay (21). This reduction by a factor of 2000 is consistent with previous data (16, 42). LPS from V. cholerae O139gave two dense silver-stained bands (41) in 16.5% tricine SDS-PAGE (31) with Mᵣ values of approximately 4,000 and 6,200 (FIG. 2A). LPS from V. cholerae O1 gave two bands with Mᵣ values of 4,000 and 15,000 (FIG. 2A). This is consistent with the observation that O139 O-SP has one hexasaccharide unit (12) whereas O1 O-SP has 12-18 repeating monosaccharide units (27). In 10% SDS-PAGE (FIG. 2B), in gels treated with Alcian Blue prior to silver staining, O139 LPS gave one band with a smear at the bottom of the gel and O139 CP gave two bands with Mᵣ values of 100,000 and 200,000, consistent with the polymerized structure of this polysaccharide (28, 36). Both V. cholerae O 139 LPS and CP were recognized by an anti-O 139 hyperimmune mice serum in immunoblotting experiments (FIG. 2C). This is consistent with the observation that O139 O-SP shares an epitope with O139 CP (43). This hyperimmune mouse serum did not react with V. cholerae O1 LPS in the same conditions. Monoclonal Ab (mAb), prepared as previously described (6), were screened by ELISA against purified O139 LPS and checked for specificity by immunoblot analysis against O139 and O1 LPS, and by agglutination with V. cholerae O139 and O1 bacterial cells. Clone B-16-5, IgM class, was selected for its high avidity to O139 pmLPS and O139 CP, as determined by ELISA inhibition. Double immunodiffusion assay showed a single band of precipitate between LPS, pmLPS, CP, and the B-16-5 mAb (FIG. 3). That pmLPS yielded a line of identity with LPS suggests that the O-139-specific antigenic determinant was preserved during the purification of the pmLPS. No cross-reactivity was observed with LPS from V. cholerae O1, serotype Inaba. The ¹H and ³¹p NMR spectra of the pmLPS, recorded on a Bruker AC 300P spectrometer, were identical to those previously reported (11). The ¹H NMR spectrum confirmed the absence of small organic molecules.

### EXAMPLE 2

### Preparation and Characterization of Vibrio cholerae O139 Conjugate.

Bacterial Strain

V. cholerae O139, strain MO45, isolated in 1992 from a patient in Madras (India), was kindly provided by Y. Takeda (Kyoto University, Japan). This strain was used for preparation of O139 antigens.

Preparation of LPS and pmLPS

Bacteria were grown in Tryptic Soy Agar (Difco, Detroit, Mich.) in Roux flasks at 37° C. for 18 h. Cells were resuspended in distilled water and LPS was obtained by hot phenol water extraction (47), followed by enzymatic treatment (DNase, RNase and protease) and ultracentrifugation (100,000 g for 3 h). The supernatant of ultracentrifugation was stored at −20° C. The pellet, containing the LPS, was dialyzed against distilled water and freeze-dried. This preparation contained 0.5% (w/v) protein and less than 0.2% (w/v) nucleic acids. LPS was treated with acetic acid to hydrolyse the lipid A-core linkage (FIG. 1) (19). LPS (10 mg/ml in 1% (v/v) aqueous acetic acid) was heated at 100° C. for 60 min. Precipitated lipid A was removed by low-speed centrifugation (350 g for 10 min). The supernatant was extracted with equal volume of chloroform-ethanol (2:1). The reaction mixture was shaken vigorously and centrifuged at 10,000 g for 30 min. The aqueous phase was dialyzed against distilled water to remove ethanol and then freeze-dried. The resulting product is referred to as pmLPS.

Derivatization and Conjugation of pmLPS

pmLPS was derivatized with adipic acid dihydrazide (ADH) as described for Haemophilus influenzae b and Shigella dysenteriae polysaccharides (7, 22, 29). Polysaccharide (5 mg/ml in 0.2 M NaCl) was brought to pH 10.75 with 0.1 M NaOH, and an equal amount of cyanogen bromide (10 mg/ml in acetonitrile) was added. The mixture was incubated for 6 min on ice and the pH was maintained at 10.75 with 0.1 M NaOH in a pHStat 719S (Metrohm, Herisau, Switzerland). An equal volume of 0.8 M ADH in 0.5 M NaHCO₃ was added and the pH was adjusted to 8.5 with 0.1 M HCl for 3.5 hours at 4° C. with the pHStat. Then, the reaction mixture was stirred overnight at 4° C. and dialyzed against demineralized water at the same temperature for 3 days. The contents of dialysis bag were freeze-dried, reconstituted in ultrapure water, passed through a P-10 Sephadex column and the void volume fractions were pooled and freeze-dried.

The derivatized pmLPS (pmLPS-AH) was dissolved in 0.2 M NaCl at 5 mg/ml. An equal weight of TT (Pasteur-Mérieux, Marcy-l'Etoile, France) was added and the pH was adjusted at 5.3 with 0.1M HCl. 1-Ethyl-3-(3-dimethylaminopropyl)-carbodiimide (EDAC) was added to a final concentration of 0.05 M and the pH was maintained with the pHStat for 4 hours at 4° C. The reaction mixture was dialyzed against PBS at 4° C. for 2 days and then passed through a column (1.5 by 90 cm) of CL-6 B Sepharose in PBS. TT was detected by measuring the optical density at 280 nm and polysaccharide by determining the refractive index.

The extent of derivatization of the activated pmLPS was calculated as the ratio ADH/polysaccharide and was 5.2% (mol/mol). For the conjugate, the pmLPS/protein (wt/wt) ratio was 1.90%, corresponding to a 0.99 mol/mol ratio. The yield was 9.6%, as calculated by the ratio of the amount of the saccharide in the conjugate over the initial amount of derivatized polysaccharide. In double-immunodiffusion assays, mAb B-16-5 gave a line of identity with pmLPS, derivatized pmLPS and TT-pmLPS, suggesting that the O139 antigenic determinant common to O-SP and CP was preserved during the conjugation of the pmLPS (FIG. 3).

Characterization of Polysaccharide Preparations

Double immunodiffusion was performed in 1% (w/v) agarose (Indubiose® IBF, Villeneuve-la-Garenne, France) in NaCl 0.5 M with 0.05% (w/v) NaN₃. Protein concentration was determined by Lowry's assay, using bovine serum albumin as the standard. The residual LPS, assayed by the Limulus amoebocyte Lysate (LAL) assay (Bio-Whittaker, Walkersville, Md.) is expressed in endotoxin units relative to the US standard (21). Nucleic acids were detected electrophoretically by using 1% agarose plate with μ DNA hydrolyzed by HindIII as the standard. LPS, pmLPS and CP were analysed by 10% SDS-PAGE and either stained with 0.5% (w/v) Alcian Blue (9) prior to silver staining (41) or electrotransferred to nitrocellulose for immunoblot analysis. LPS and pmLPS were analyzed by Tricine SDS-PAGE (31, 49) using a 16.5% (w/v) running gel and a 4% stacking gel and silver stained. The ¹H and ³¹P NMR spectra of the pmLPS were recorded on a Bruker AC 300P spectrometer.

### EXAMPLE 3

### Preparation of Vibrio cholerae O1 Conjugate

Bacterial Strain

V. cholerae O1 CNRVC 950707, serotype Inaba strain isolated in 1995 from a patient in Mali, was used for preparation of O1 LPS.

Preparation of LPS and pmLPS

Same methods as described for V. cholerae O139 (see example 2)

Derivatization and Conjugation of pmLPS

The only modification concerned the temperature of the incubation steps of the reagents in the pHStat which was room temperature for O1 conjugate instead of +4° C. as previously described for O139 conjugate (see example 2).

### EXAMPLE 4

### Anti-O139 and anti-TT Abs Response of Mice

Immunization

Six week old female BALB/c were injected subcutaneously with 2.5 μg of pmLPS O139 alone, or as a conjugate (see example 2), as described in the footnote of Table I. A group of mice was immunized similarly with 2.5 μg of TT. LPS and TT Ab levels were determined by ELISA. Plates were coated with either LPS or TT. Serial twofold dilutions of mouse sera (1/100 to 1/6,400) were analyzed. The secondary Abs used were either peroxidase-conjugated anti-mouse IgG (γ chain-specific), or IgM (μ chain-specific). The results were calculated for each immunoglobulin class, as a percent of a high-titered reference serum arbitrarily assigned a value of 100 ELISA units (EU) by parallel line analysis with a program from the Centers for Disease Control and expressed as the geometric mean (35). Following the same method, anti-TT Ab level was expressed with respect to an hyperimmune mouse pooled standard serum prepared by repeated immunizations of mice with TT. Serum anti-O139 Ab titers are shown in Table I. Pre-immune sera and PBS control sera contained no detectable levels of Abs. After the second immunization, pmLPS elicited a moderate IgM response and a very weak IgG response, consistent with the response induced by a T-independent antigen. After the third immunization with pmLPS-TT, IgM titers were equivalent to those elicited in response to pmLPS. After the fourth immunization, pmLPS-TT elicited a very much higher IgG response than pmLPS (P=0.0011), lasting at least 231 days (P=0.0046). This IgG response demonstrates a booster effect and an immunoglobulin isotype switch. This strongly suggests that the pmLPS was functionally converted, due to the protein carrier effect, into a T-dependent antigen. In inhibition ELISA (42), the binding of anti-pmLPS-TT antibodies to O139 LPS was inhibited by either O139 LPS (amount of antigen yielding 50% inhibition: 8 μg/ml) or O139 CP (1 μg/ml). Serum anti-TT Ab titers are shown in Table I. Pre-immune sera contained no detectable levels of anti-TT Abs. After the third immunization, pmLPS-TT elicited a significant increase in anti-TT IgG levels (P<0.01), similar to that in mice immunized with TT alone.

Vibriocidal Abs Response

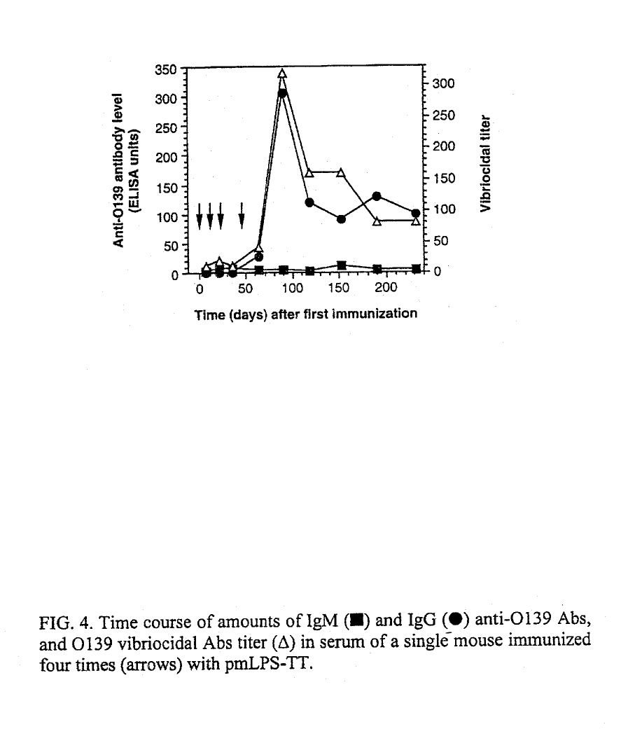

The vibriocidal tests were performed as previously described (5) with two-fold dilutions (beginning with an initial 1:10 dilution) using V. cholerae O139 strain MO10-T4, a spontaneous nonencapsulated variant of MO10 (43), kindly provided by A. Weintraub (Karolinska Institute, Huddinge, Sweden), as the target strain and Guinea pig serum as the source of complement. The vibriocidal titer was defined as the reciprocal of the highest dilution of serum causing 100% bacterial lysis. Controls for each assay included, in addition to the usual cell control and complement control, a positive hyperimmune control serum with a titer of 1/2560. Consecutive sera of one mouse immunized with pmLPS-TT were tested for vibriocidal activity (FIG. 4). There was a correlation between the kinetics of the vibriocidal Abs titer and the anti-O139 IgG level (correlation coeficient=0.89). Findings for sera from other mice immunized with pmLPS-TT supported this correlation.

Protective Activity of Anti-pmLPS-TT Abs

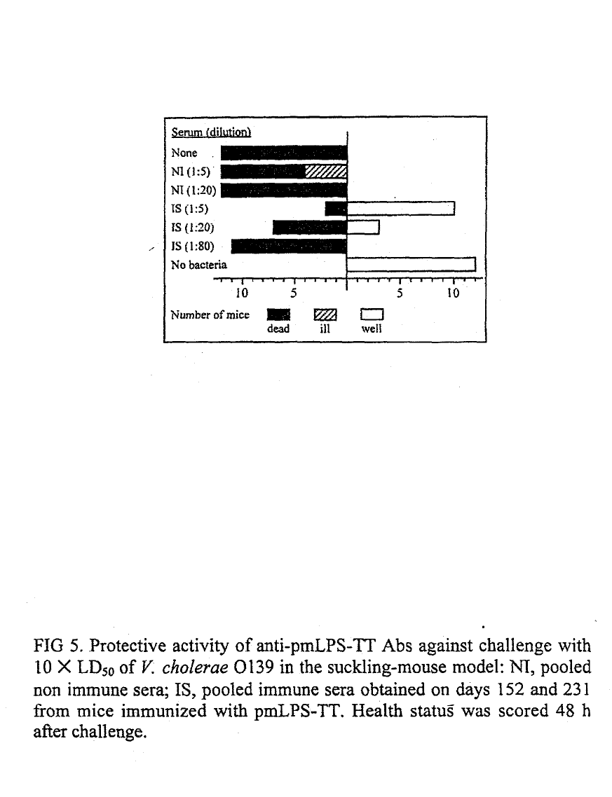

Suckling Swiss mice 5 days old and weighing 3.3 to 4.4 g were used for oral challenge experiments with V. cholerae O139. A V. cholerae O139 strain, isolated in 1992 from a patient in India and selected for its capacity to produce high levels of cholera toxin (5 μg/ml), was used for oral challenge in mice. After removing secreted cholera toxin, a dose of 3.5×10⁸ V. cholerae cells (10 times the 50% lethal dose), preincubated for 30 min at 37° C. with immune serum at various dilutions in 0.1 ml, was delivered into the stomach with a blunt-tip feeding needle. Groups of mice that received vibrio suspension alone, PBS alone or Vibrio suspension with non-immunized mice serum served as controls. Mice were maintained at 30° C. for 48 h or until death, and all surviving mice were scored as well or ill at 48 h. Mice were considered ill if they met all of the following criteria: diarrhea, markedly reduced skin turgor and poor response to stimuli. Mice that received pooled immune sera, collected on days 152 and 231 from mice immunized with pmLPS-TT, diluted 1:5, were significantly protected (FIG. 5). The level of protection decreased as the dilution of the pooled immune sera increased: protection was therefore dependent on dose. No protection was observed in mice that received pooled non-immune control sera.

TABLE I
Geometric mean ELISA titer (25th-75th percentiles) of serum anti-LPS and anti-TT Abs elicited in mice
following immunization with pmLPS alone or as a conjugate^{a}

| | Abs and immunogen | | | | | |
|---|---|---|---|---|---|---|
| | Anti-TT IgG | Anti-LPS IgM | | Anti-LPS IgG | | |
| Day^{b} | pmLPS-TT | pm-LPS | pmLPS-TT | pm-LPS | pmLPS-TT | P^{c} |
| 0* | <1 | <1 | <1 | <1 | <1 | NS^{d} |
| 7 | <1 | 2.8 (2.3-3.5) | 1.8 (1.5-2.2) | 1.4 (1.3-1.5) | 1.2 (0.9-1.6) | NS |
| 14* | | | | | | |
| 21 | 9.3 (5.3-12.2) | 7.9 (4.8-14.2) | 6.2 (5.4-8.8) | <1 | 1 (0.8-1) | NS |
| 28* | | | | | | |
| 35 | 61.9 (52.8-109.5) | 17.5 (7.5-41.8) | 17.7 (12.3-24.3) | <1 | 1.8 (0.9-4.6) | NS |
| 56* | | | | | | |
| 63 | 193.5 (155.9-219.2) | 6.5 (3.6-11.4) | 8.1 (6.2-9) | 2 (1.2-4.9) | 11.7 (8.1-15.3) | 0.0011 |
| 91 | 280.5 (204.1-454.8) | 4.2 (3.1-6.8) | 7.5 (5.7-8.8) | <1 | 36.3 (11.7-67.3) | 0.0357 |
| 119 | 191.3 (156.4-308.8) | 4.4 (2.5-6.9) | 2.8 (2-3.5) | 1.2 (1.1-3.3) | 21.4 (7.3-45.8) | 0.0249 |
| 152 | 209.5 (181.1-295.3) | 4.2 (2.9-6.4) | 12.7 (10.1-15.3) | 2.5 (2.1-3.1) | 29.9 (14.8-78.3) | 0.0131 |
| 190 | 192.2 (147.9-277.9) | 4.6 (3.5-6.6) | 7.2 (6-9.5) | 1.3 (1.1-2.2) | 30.6 (15.7-72.3) | 0.0055 |
| 231 | 183^{e} (144.4-249.5) | 4.5 (3.1-6.5) | 6.3^{e} (4.5-9.3) | <1 | 23.8^{e} (12.3-60.6) | 0.0046 |

^{a}Ten mice were injected subcutaneously with saline solutions containing 2.5 μg of the antigen three times at 2 weeks intervals and were then given a fourth injection 4 weeks later. The mice were bled 7 days after each injection and then again each month for 6 months after the fourth injection.
^{b}Days of immunization are marked with an asterisk.
^{c}Comparison of titers of anti-LPS IgG elicited by pmLPS-TT versus pmLPS (P values are calculated by Student's t test).
^{d}NS, not significant.
^{e}Nine mice were tested.

### EXAMPLE 5

### Results Obtained with the V. cholerae O1 Conjugate

Mice were immunized as previously described for V. cholerae O139 conjugate. Serum anti-O1 antibodies titer at days 7 and 68 after the first immunization are shown in Table II.

TABLE II
ELISA titers of serum anti-V. cholerae O1 LPS antibodies
in mice 7 days and 68 days after immunization
with V. cholerae O1 conjugate^{a}

| | ELISA titer | | | |
|---|---|---|---|---|
| | Anti-LPS IgM | | Anti-LPS IgG | |
| Mice | Day 7 | Day 68 | Day 7 | Day 68 |
| Num- | after | after | after | after |
| ber | immunization | Immunization | immunization | immunization |
| 1 | <1 | 4 | 2 | 6 |
| 2 | <1 | 5 | 1 | 82 |
| 3 | <1 | 6 | 2 | 13 |
| 4 | <1 | 6 | 4 | 18 |
| 5 | <1 | 7 | 2 | 13 |
| 6 | <1 | 4 | 2 | 7 |
| 7 | <1 | 3 | 2 | 18 |
| 8 | <1 | 13 | 2 | 36 |
| 9 | <1 | 4 | 2 | 14 |
| 10 | <1 | 9 | 3 | 7 |

^{a}Mice were injected subcutaneously with saline solution containing 2.5 μg of the conjugate three times at 2-weeks interval and were given a fourth injection 4 weeks later. The mice were bleed at days 7 and 68 after the first immunization.

The V. cholerae O1 conjugate elicited high levels of IgG antibodies compared to low levels of IgM. Conjugation of the V. cholerae O1 polysaccharide, therefore, conferred T-dependent properties on this polysaccharide.

### REFERENCES

1. Albert, M. J. 1994.Vibrio cholerae O139 Bengal. J. Clin. Microbiol. 32:2345-2349.

2. Albert, M. J., K. Alam, A. S. M. H. Rahman, S. Huda, and R. B. Sack. 1994. Lack of cross-protection against diarrhea due to Vibrio cholerae O1 after oral immunization of rabbits with V. cholerae O139 Bengal. J. Infect. Dis. 169:709-710.

3. Berche, P., C. Poyart, E. Abachin, H. Lelievre, J. Vandepitte, A. Dodin, and J. M. Fournier. 1994. The novel epidemic strain O139 is closely related to the pandemic strain O1 of Vibrio cholerae . J. Infect. Dis. 170:701-704.

4. Bondre, V. P., V. B. Sinha, and B. S. Srivastava. 1998. Evaluation of different subcellular fractions of Vibrio cholerae O139 in protection to challenge in experimental cholera. FEMS Immunol. Med. Microbiol. 19:323-329.

5. Bougoudogo, F., F. Vely, F. Nato, A. Boutonnier, P. Gounon, J. C. Mazié, and J. M. Fournier. 1995. Protective activities of serum immunoglobulin G on the mucosal surface to Vibrio cholerae O1. Bull. Inst. Pasteur 93:273-283.

6. Boutonnier, A., F. Nato, A. Bouvet, L. Lebrun, A. Audurier, J. C. Mazié, and J. M. Fournier. 1989. Direct testing of blood cultures for detection of the serotype 5 and 8 capsular polysaccharides of Staphylococcus aureus. J. Clin. Microbiol. 27:989-993.

7. Chu, C. Y., B. K. Liu, D. Watson, S. S. Szu, D. Bryla, J. Shiloach, R. Schneerson, and J. B. Robbins. 1991. Preparation, characterization, and immunogenicity of conjugates composed of the O-specific polysaccharide of Shigella dysenteriae type 1 (Shiga's bacillus) bound to tetanus toxoid. Infect. Immun. 59:4450-4458.

8. Comstock, L. E., J. A. Johnson, J. M. Michalski, J. G. Morris, and J. B. Kaper. 1996. Cloning and sequence of a region encoding a surface polysaccharide of Vibrio cholerae O139 and characterization of the insertion site in the chromosome of Vibrio cholerae O1. Mol. Microbiol. 19:815-826.

9. Corzo, J., R. Pérez-Galdona, M. Leon-Barrios, and A. M. Gutiérrez-Navarro. 1991. Alcian Blue fixation allows silver staining of the isolated polysaccharide component of bacterial lipopolysaccharides in polyacrylamide gels. Electrophoresis 12:439-441.

10. Coster, T. S., K. P. Killeen, M. K. Waldor, D. T. Beattie, D. R. Spriggs, J. R. Kenner, A. Trofa, J. C. Sadoff, J. J. Mekalanos, and D. N. Taylor. 1995. Safety, immunogenicity, and efficacy of live attenuated Vibrio cholerae O139 vaccine prototype. Lancet 345:949-952.

11. Cox, A. D., J. R. Brisson, V. Varma, and M. B. Perry. 1996. Structural analysis of the lipopolysaccharide from Vibrio cholerae O139. Carbohydr. Res. 290:43-58.

12. Cox, A. D., and M. B. Perry. 1996. Structural analysis of the O-antigen-core region of the lipopolysaccharide from Vibrio cholerae O139. Carbohydr. Res. 290:59-65.

13. Devi, S. J. N., U. Hayat, C. E. Frasch, A. S. Kreger, and J. G. Morris. 1995. Capsular polysaccharide-protein conjugate vaccines of carbotype 1 Vibrio vulnificus: construction, immunogenicity, and protective efficacy in a murine model. Infect. Immun. 63:2906-2911.

14. Dumontier, S., and P. Berche. 1998. Vibrio cholerae O22 might be a putative source of exogenous DNA resulting in the emergence of the new strain of Vibrio cholerae O139. FEMS Microbiol. Lett. 164:91-98.

15. Faruque, S. M., A. K. Siddique, M. N. Saha, Asadulghani, M. M. Rahman, K. Zaman, M. J. Albert, D. A. Sack, and R. B. Sack. 1999. Molecular characterization of a new ribotype of Vibrio cholerae O139 Bengal associated with an outbreak of cholera in Bangladesh. J. Clin. Microbiol. 37:1313-1318.

16. Gupta, R. K., S. C. Szu, R. A. Finkelstein, and J. B. Robbins. 1992. Synthesis, characterization, and some immunological properties of conjugates composed of the detoxified lipopolysaccharide of Vibrio cholerae O1 serotype Inaba bound to cholera toxin. Infect. Immun. 60:3201-3208.

17. Gupta, R. K., D. N. Taylor, D. A. Bryla, J. B. Robbins, and S. S. C. Szu. 1998. Phase 1 evaluation of Vibrio cholerae O1, serotype Inaba, polysaccharide-cholera toxin conjugates in adult volunteers. Infect. Immun. 66:3095-3099.

18. Habeeb, A. F. 1966. Determination of free amino groups in proteins by trinitrobenzenesulfonic acid. Anal. Biochem. 14:328-336.

19. Hancock, I. C., and I. R. Poxton. 1988. Appendix 1. General methods, p. 269-286. In I. C. Hancock, and I. R. Poxton (ed.), Bacterial cell surface techniques. John Wiley & Sons, Chichester, UK.

20. Herbert, D., P. J. Phipps, and R. E. Strange. 1971. Chemical analysis of microbial cells. Meth. Microbiol. 5B:209-344.

21. Hochstein, H. D. 1990. Role of the FDA in regulating the Limulus amoebocyte lysate test, p. 38-49. In R. B. Prior (ed.), Clinical applications of the Limulus amoebocyte lysate test. CRC Press, Boca Raton, FL.

22. Jennings, H. J., and R. K. Sood. 1994. Synthetic glycoconjugates as human vaccines, p. 325-371. In Y. C. Lee, and R. T. Lee (ed.), Neoglycoconjugates: preparation and applications. Academic Press, San Diego, Calif.

23. Jertborn, M., A. M. Svennerholm, and J. Holmgren. 1996. Intestinal and systemic immune responses in humans after oral immunization with a bivalent B subunit-O1/O139 whole cell cholera vaccine. Vaccine 14:1459-1465.

24. Johnson, J. A., A. Joseph, and J. G. Morris. 1995. Capsular polysaccharide-protein conjugate vaccines against Vibrio cholerae O139 Bengal. Bull. Inst. Pasteur 93:285-290.

25. Jonson, G., J. Osek, A. M. Svennerholm, and J. Holmgren. 1996. Immune mechanisms and protective antigens of Vibrio cholerae serogroup O139 as a basis for vaccine development. Infect. Immun. 64:3778-3785.

26. Kabir, S. 1982. Characterization of the lipopolysaccharides from Vibrio cholerae O395 (Ogawa). Infect. Immun. 38:1263-1272.

27. Kenne, L., B. Lindberg, P. Unger, B. Gustasfson, and T. Holme. 1982. Structural studies of the Vibrio cholerae O-antigen. Carbohydr. Res. 100:341-349.

28. Knirel, Y. A., L. Paredes, P. E. Jansson, A. Weintraub, G. Widmalm, and M. J. Albert. 1995. Structure of the capsular polysaccharide of Vibrio cholerae O 139 synonym Bengal containing D-galactose 4,6-cyclophosphate. Eur. J. Biochem. 30 232:391-396.

29. Konadu, E., J. B. Robbins, J. Shiloach, D. A. Bryla, and S. C. Szu. 1994. Preparation, characterization, and immunological properties in mice of Escherichia coli O157 O-specific polysaccharide-protein conjugate vaccines. Infect. Immun. 62:5048-5054.

30. Kossaczka, Z., J. Shiloach, V. Johnson, D. N. Taylor, R. A. Finkelstein, J. B. Robbins, and S. C. Szu. 2000.Vibrio cholerae O139 conjugate vaccines: synthesis and immunogenicity of V cholerae O139 capsular polysaccharide conjugates with recombinant diphtheria toxin mutant in mice. Infect. Immun. 68:5037-5043.

31. Lesse, A. J., A. A. Campagnari, W. E. Bittner, and M. A. Apicella. 1990. Increased resolution of lipopolysaccharides and lipooligosaccharides utilizing tricine-sodium dodecyl sulfate-polyacrylamide gel electrophoresis. J. Immunol. Meth. 126:109-117.

32. Mukholpadhyay, A. K., A. Basu, P. Garg, P. K Bag, A. Ghosh, S. K Bhattacharya, Y. Takeda, and G. B. Nair. 1998. Molecular epidemiology of reemergent Vibrio cholerae O139 Bengal in India. J. Clin. Microbiol. 36:2149-2152.

33. Nandy, R. K, M. J. Albert, and A. C. Ghose. 1996. Serum antibacterial and antitoxin responses in clinical cholera caused by Vibrio cholerae O139 Bengal and evaluation of their importance in protection. Vaccine 14:1137-1142.

34. Nandy, R. K., S. Mukhopadhyay, A. N. Ghosh, and A. C. Ghose. 1999. Antibodies to the truncated (short) form of ‘O’ polysaccharides (TFOP) of Vibrio cholerae O139 lipopolysaccharides protect mice against experimental cholera induced by encapsulated O139 strains and such protection is mediated by inhibition of intestinal colonization of vibrios. Vaccine 17:2844-2852.

35. Plikaytis, B. D., P. F. Holder, and G. M. Carlone. 1996. Program ELISA for Windows user's manual, version 1.00, Centers for Disease Control and Prevention, Atlanta, Ga.

36. Preston, L. M., Q. W. Xu, J. A. Johnson, A. Joseph, D. R. Maneval, K. Husain, G. P. Reddy, C. A. Bush, and J. G. Morris. 1995. Preliminary structure determination of the capsular polysaccharide of Vibrio cholerae O139 Bengal A11837. J. Bacteriol. 177:835-838.

37. Reuhs, B. L., R. W. Carlson, and J. S. Kim. 1993.

Rhizobium fredii and Rhizobium meliloti produce 3-deoxy-D-manno-2-octulosonic acid-containing polysaccharides that are structurally analogous to group II K antigens (capsular polysaccharides) found in Escherichia coli. J. Bacteriol. 175:3570-3580.

38. Robbins, J. B., R. Schneerson, and S. C. Szu. 1995. Hypothesis: serum IgG antibody is sufficient to confer protection against infectious diseases by inactivating the inoculum. J. Infect. Dis. 171:1387-1398.

39. Sengupta, D. K, M. Boesman-Finkelstein, and R. A. Finkelstein. 1996. Antibody against the capsule of Vibrio cholerae O139 protects against experimental challenge. Infect. Immun. 64:343-345.

40. Tacket, C. O., G. Losonsky, J. P. Nataro, L. Comstock, J. Michalski, R. Edelman, J. B. Kaper, and M. M. Levine. 1995. Initial clinical studies of CVD 112 Vibrio cholerae O139 live oral vaccine: safety and efficacy against experimental challenge. J. Infect. Dis. 172:883-886.

41. Tsai, C. M., and C. E. Frasch. 1982. A sensitive silver stain for detecting lipopolysaccharides in polyacrylamide gels. Anal. Biochem. 119:115-119.

42. Villeneuve, S., A. Boutonnier, L. A. Mulard, and J. M. Fournier. 1999. Immunochemical characterization of an Ogawa-Inaba common antigenic determinant of Vibrio cholerae O1. Microbiology 145:2477-2484.

43. Waldor, M. K., R. Colwell, and J. J. Mekalanos. 1994. The Vibrio cholerae O139 serogroup antigen includes an O-antigen capsule and lipopolysaccharide virulence determinants. Proc. Natl. Acad. Sci. USA 91:11388-11392.

44. Waldor, M. K., and J. J. Mekalanos. 1994. Emergence of a new cholera pandemic: molecular analysis of virulence determinants in Vibrio cholerae O139 and development of a live vaccine prototype. J. Infect. Dis. 170:278-283.

45. Wang, D., and E. Kabat. 1996. Carbohydrate antigens (polysaccharides), p. 247-276. In M. H. V. Van Regenmortel (ed.), Structure of antigens, vol. 3. CRC Press, New York.

46. Weintraub, A., G. Widmalm, P. E. Jansson, M. Jansson, K. Hultenby, and M. J. Albert. 1994. Vibrio cholerae O139 Bengal possesses a capsular polysaccharide which may confer increased virulence. Microb. Pathog. 16:235-241.

47. Westphal, O., and K. Jann. 1965. Bacterial lipopolysaccharide extraction with phenol-water and further applications of procedure, p. 83-91. In R. L. Wistler (ed.), Methods in Carbohydrates Chemistry, vol. 5. Accademic Press Inc., New York.

48. Wilkinson, S. G. 1996. Bacterial lipopolysaccharides: themes and variations. Prog. Lipid Res. 35:283-343.

49. Schägger, H., and G. von Jagow. 1987. Tricine-sodium dodecyl sulfate-polyacrylamide gel electrophoresis for the separation of proteins in the range from 1 to 100 kDa. Anal. Biochem. 166:368-379.

1. An immunogenic composition against Vibrio infection comprising an O-SP unit of LPS of Vibrio associated to a core molecule of LPS of Vibrio or a polymer of said composition.

2. The immunogenic composition of claim 1, wherein the O-SP unit associated to the core molecule of LPS of Vibrio is part of a conjugate further comprising a carrier protein.

3. The immunogenic composition of claim 2, wherein the Vibrio O-SP unit and core molecule are bound to the carrier protein of the conjugate by a covalent link.

4. The immunogenic composition of claim 2, wherein the carrier protein is a bacterial protein.

5. The immunogenic composition of claim 4, wherein the bacterial protein is tetanus toxoid.

6. The immunogenic composition of claim 1, wherein said composition further comprises an adjuvant and/or a pharmaceutically acceptable carrier.

7. The immunogenic composition of claim 1, wherein the LPS is from Vibrio cholera.

8. The immunogenic composition of claim 1, wherein the LPS is from Vibrio cholera serogroup O139.

9. A vaccine composition protective against infection from Vibrio wherein said vaccine composition comprises an immunogenic composition according to claim 1.

10. The vaccine composition of claim 9, wherein said vaccine composition is protective against infection from Vibrio cholerae.

11. The vaccine composition of claim 10, wherein said vaccine composition is protective against infection from Vibrio cholerae serogroup O139.

12. A method for preparing a conjugate comprising an O-SP unit of LPS from a Vibrio associated to a core molecule of LPS of a Vibrio bound to a protein carrier, said method comprising:
a) providing LPS from a Vibrio;
b) hydrolyzing the lipid A-core linkage for obtaining an O-SP unit associated to a core molecule;
c) derivatizing the O-SP unit associated to the core molecule of step b);
d) bounding the derivatized the O-SP unit associated to the core molecule of step c) to a carrier protein;
e) collecting the O-SP unit associated to the core molecule bound to the carrier protein in step d.

13. The method of claim 12, wherein the O-SP unit associated to the core molecule are bound to the carrier protein by a covalent link.

14. The method of claim 12, wherein the carrier protein is a bacterial protein.

15. The method of claim 14, wherein the bacterial protein is tetanus toxoid.

16. The method of claim 12, wherein LPS of step a) is from Vibrio cholerae.

17. The method of claim 16, wherein LPS of step a) is from Vibrio cholerae serogroup O139.

18. Use of a composition comprising a conjugate compound comprising an O-SP unit of LPS of Vibrio associated to a core of LPS of Vibrio bound to a protein carrier for the preparation of a medicament for preventing a Vibrio infection.

19. Use of claim 18, wherein Vibrio infection is an infection from Vibrio cholerae.

20. Use of claim 19, wherein Vibrio cholerae infection is an infection from Vibrio cholerae serogroup O139.

21. A conjugate compound comprising an O-SP unit of LPS of Vibrio associated to a core molecule of LPS of Vibrio bound to a protein carrier.

22. The conjugate compound of claim 21, wherein the Vibrio O-SP unit associated to the Vibrio core molecule is bound to the protein carrier by a covalent link.

23. The conjugate compound of claim 21, wherein the protein carrier is a bacterial toxin.

24. The conjugate compound of claim 23, wherein the bacterial toxin is tetanus toxoid.

25. The conjugate compound of claim 21, wherein the Vibrio LPS is from Vibrio cholerae.

26. The conjugate compound of claim 25, wherein the Vibrio cholerae LPS is from Vibrio cholerae serogroup O139.

27.The composition of claim 1 wherein the O-SP unit and the core are from two different Vibrio.

28.The conjugate of claim 21, wherein the O-SP unit and the core are from two different Vibrio.

29. Method for immunizing human or animal against Vibrio infection, wherein said method comprises administration to said human or animal of a composition as defined hereabove, wherein Vibrio infection is preferably an infection from Vibrio cholerae and more preferably from Vibrio cholerae serogroup O 139.
